(19) United States
(12) Patent Application Publication
(10) Pub. No.: US 2007/0027463 A1
(43) Pub. Date: Feb. 1, 2007

(54) Cutting Device and Method of Use

(76) Inventor: Michael T. Neubig, LaJolla, CA (US)

Correspondence Address: PROCOPIO, CORY, HARGREAVES & SAVITCH LLP, 530 B STREET, SUITE 2100, SAN DIEGO, CA, 92101, US

(21) Appl. No.: 11/425,075

(22) Filed: Jun. 19, 2006

Related U.S. Application Data
(60) Provisional application No. 60/696,435, filed on Jun. 29, 2005.

(51) Int. Cl. A61B 17/32

(52) U.S. Cl. 606/167

(57) ABSTRACT

A cutting device for cutting through biological tissue at a controlled depth without damaging underlying tissue includes a holding member to be held by a user for operating the cutting device; a cutting blade coupled to the holding member, the cutting blade including a cutting edge for cutting through the biological tissue; and a guide surface opposite of and fixed relative to the cutting edge to maintain a controlled cutting depth without damaging underlying tissue.

### CROSS-REFERENCE TO RELATED APPLICATIONS

This application claims the benefit of U.S. Provisional Application 60/696,435 filed Jun. 29, 2005 under 35 U.S.C. 119(e).

### FIELD OF THE INVENTION

The present invention is, in general, in the field of cutting devices, and, in particular, in the field of cutting devices for cutting biological tissue at a controlled depth to prevent damage to underlying tissue.

### BACKGROUND OF THE INVENTION

In surgery, animal experiments, and other applications, it is sometimes desirable to cut through biological tissue with a blade without disturbing or damaging underlying tissue. For example, but not by way of limitation, in certain animal experiments, it is desirable to access the brain of a juvenile animal such as in brain experimentation on juvenile rodents. To access the brain of the juvenile rodent, the skull may be cut open with scissors in which case the user has to be extremely careful when first piercing the skull, then when opening the scissors a small amount, and then when closing the scissors a small amount (not all the way). The small distance between the skull and the brain make this a difficult procedure to learn and carry out without damaging the brain. The same problem is true for the use of a surgical scalpel to cut the skull.

### SUMMARY OF THE INVENTION

To solve these problems and others, the present invention relates to a cutting device for cutting biological tissue at a controlled depth to prevent damage to underlying tissue. In an aspect of the invention, the cutting device includes a holding member to be held by a user for operating the cutting device; a cutting blade coupled to the holding member, the cutting blade including a cutting edge for cutting through the biological tissue; and a guide surface opposite of and fixed relative to the cutting edge to maintain a controlled cutting depth without damaging underlying tissue.

Another aspect of the invention involves a method of cutting through biological tissue at a controlled depth without damaging underlying tissue. The method includes providing a cutting device including a holding member to be held by a user, a cutting blade with a cutting edge, cutting angle, and vertex coupled to the holding member, the cutting blade including a cutting edge, cutting angle, and vertex, and a guide surface opposite of and fixed relative to the cutting edge; inserting the cutting edge of the cutting device through the biological tissue; and moving the cutting device so that the cutting edge of the blade cuts the biological tissue without damaging underlying tissue and the guide surface remains tangent with the biological tissue adjacent to the vertex.

A further aspect of the invention involves a cutting device including a holding member to be held by a user for operating the cutting device; a cutting blade coupled to the holding member, the cutting blade including a cutting edge; and a guide surface opposite of and fixed relative to the cutting edge to maintain a controlled cutting depth.

A still further aspect of the invention involves a method of cutting through an object at a controlled depth. The method includes providing a cutting device including a holding member to be held by a user, a cutting blade coupled to the holding member, the cutting blade including a cutting edge, cutting angle, and vertex, and a guide surface opposite of and fixed relative to the cutting edge; at least one of inserting the cutting edge of the cutting device through the object or positioning the cutting edge of the cutting device adjacent an edge of the object; and moving the cutting device so that the cutting edge of the blade cuts the object and the guide surface remains tangent with the object adjacent to the vertex.

Further objects and advantages will be apparent to those skilled in the art after a review of the drawings and the detailed description of the preferred embodiments set forth below.

### BRIEF DESCRIPTION OF THE DRAWINGS

FIG. 1 is a top perspective view of an embodiment of a cutting device shown in an exemplary cutting of the soft skull of a juvenile rodent.

FIG. 2 is a front elevational view of an embodiment of the cutting device illustrated in FIG. 1.

FIG. 3 is a front elevational view of an additional embodiment of a cutting device.

FIG. 4 is a front elevational view of another embodiment of a cutting device.

FIG. 5 is a front elevational view of a further embodiment of a cutting device.

FIG. 6 is a front elevational view of the embodiment of the cutting device of FIGS. 1, 2, and 6, and is shown in use.

FIG. 7 is a front elevational view of another embodiment of a cutting device shown in use.

FIGS. 8-10 illustrate a side elevational view, a combination front elevational view and cross-sectional view, and a top plan view of an additional embodiment of a cutting device shown in use.

FIGS. 11-14 illustrate a still further embodiment of a cutting device, in which FIG. 11 is a top plan view of a spring element of the cutting device, FIG. 12 is a front elevational view of the cutting device, FIG. 13 is an enlarged front elevational view of a cutting block of the cutting device, and FIG. 14 is an enlarged side elevational view of the cutting block taken along lines 14-14 of FIG. 12.

### DETAILED DESCRIPTION OF PREFERRED EMBODIMENTS

With reference initially to FIGS. 1, 2, and 6, an embodiment of a cutting device 50 for cutting biological tissue or other materials at a controlled or defined depth to prevent damage to underlying tissue or objects will be described. In FIG. 1, the cutting device 50 is shown being used to cut the soft skull of a juvenile rodent (e.g., rat, mice, gerbil) without damaging the underlying brain in order to access the brain for further experimentation. However, the cutting device 50 (and further cutting devices described and shown herein) may be used in other cutting applications besides cutting the soft skull of juvenile animals. For example, but not by way of limitation, the cutting device(s) may be used to cut through the biological tissue of other animals (e.g., animal surgery), the biological tissue of humans (e.g., human surgery), materials other than biological tissue, the cortex of an object, an exoskeleton, a tendon sheath, and a blood vessel. As a further example, the cutting device 50 (and further cutting devices described and shown herein) may be used by hobbyists, for cutting veneer, making jigsaw puzzles, jewelry making, or other cutting applications.

In the embodiment of the cutting device 50 shown in FIGS. 1, 2, and 6, the cutting device 50 includes two main components: a cutting member 60 and a guide member 70. The cutting member 60 includes a blade 90. The blade 90 includes an upper cutting edge 100, a bottom dull edge 110, and a sharp, pointed distal tip 120, which may be used to pierce the tissue or materials when starting a cut. The guide member 70 is preferably made of a clear, transparent material so that a user can see through it and observe the tissue and cut in process. The guide member 70 includes a rear portion 130 with a handle 80, a front portion 140, an upper surface 150, and a lower guide surface 160 with a smooth surface coating (e.g., Teflon coating). As used herein, front portion 140 is the portion of the guide member 70 that the tip 120 points at and the rear portion 130 is the portion of the guide member 70 opposite the front portion 140. The handle 80 forms a holding portion that may be gripped by a user to control movement of the cutting device 50. Using the handle 80, a line of force tangent to the skull 214 is provided by the user at the point of the leading edge of the cut.

The blade 90 extends through the guide member 70 so that the cutting edge 100 and guide surface 160 are opposite of each other. The cutting edge 100 and the guide surface 160 intersect at a vertex 170 and form a fixed cutting angle CA during use. The cutting angle CA is sized to provide optimal cutting for a given application and is preferably less than 90 degrees to ensure that the guide surface 160 rides on the tissue surface. The distal tip 120 extends from the guide surface 160 a distance or depth D. Of the blade 90, the distal tip 120 preferably extends below the guide surface 160 the greatest perpendicular distance. The blade 90 is connected to the guide member 70 so that, in use, the blade 90 does not move relative to the guide member 70.

The blade 90 may be permanently fixed relative to the guide member 70, or the blade 90 may be movable/adjustable relative to the guide member for adjusting the depth D of the cutting edge 100 (i.e., the amount of the cutting edge 100 exposed), the cutting angle CA of the cutting edge 100, and/or the position of the cutting edge 100.

In a further embodiment, the blade 90 may be removable/replaceable or adjustable for different cutting applications. For example, the cutting edge 100 may be set relative to the guide member 70 at a first depth D and/or cutting angle CA for cutting the soft skull of a young animal, and may be set at a greater depth D and/or different cutting angle for cutting the skull of an older animal with a thicker and/or harder skull.

Alternatively, the cutting member 60/blade 90 may be removably attachable to the guide member 70 so that different size/configuration guide members 70 (e.g., as part of a cutting device kit) may be used with the same cutting member 60 for different cutting applications or different size/configuration cutting members 60/blades 90 may be removably attachable to the same guide member 70 for different cutting applications.

In another embodiment of a kit, the kit may include more than one of the cutting devices described herein. In such an embodiment, the cutting devices may be of the same general configuration, but designed for different applications (the cutting devices may include at least one of different cutting edges, different cutting edge angles, different cutting edge depths, and different handle member configurations), or some of the cutting devices may have different configurations.

The cutting member 60 and/or the guide member 70 may be made out of a sterilizable/autoclavable material such as, but not limited to, a metal such as stainless steel or titanium. In such an embodiment, the cutting member 60 and/or the guide member 70 may be sterilized/autoclaved between each use or after multiple uses. Alternatively, the guide member 70 may be made out of a plastic material. The cutting member 60 and/or the guide member 70 may disposable, and as such, may be disposed of after a single use or after multiple uses.

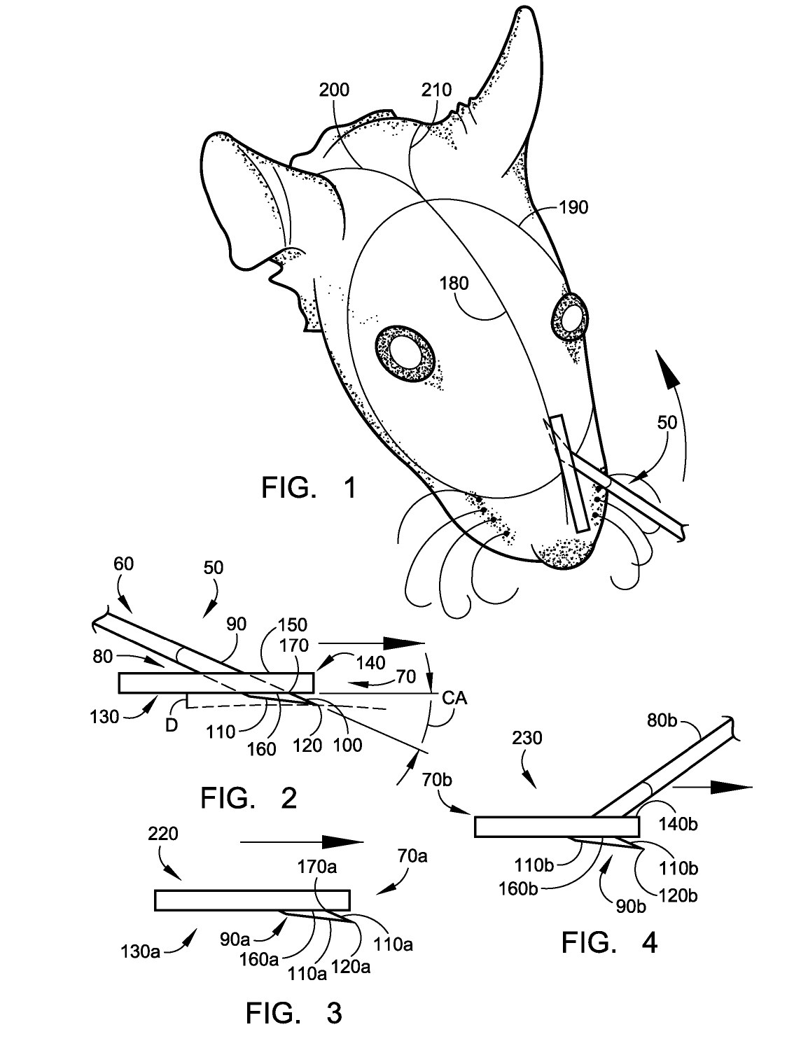

With reference to FIGS. 1 and 6, an exemplary use of the cutting device 50 will be described. As indicated above, the cutting device 50 may be used to cut a soft skull 214 of a juvenile animal such as the soft skull of rodent to access the brain for further experimentation, without damaging the brain. The sharp, pointed distal tip 120 of the cutting device 50 may be inserted/pierced through the skull 214, for example, at a point just above the nose, so that the cutting edge 100 is oriented in the direction of the desired cutting line 180, 190 and cutting travel (see arrow of FIG. 2). Alternatively, a separate instrument may be used to make the incision, and the blade 90 of the cutting device 50 may then be inserted through the incision. Upon insertion of the cutting device 50 and during use of the cutting device 50, the lower guide surface 160 of the guide member 70 controls the depth D of the blade 90, preventing the distal tip 120 from extending too deep and damaging underlying brain 216. The depth D of the distal tip 120 is less than the distance between the outer surface of the skull and the outer surface of the brain 216. After inserting the distal tip 120 and the cutting edge 100 through the skull 214, using the handle 80, a line of force tangent to the skull 214 is provided by the user at the point of the leading edge of the cut and the cutting device 50 is moved in the direction of the arrow shown in FIG. 2, in the path of the desired cutting line 180, 190 (FIG. 1). For example, if a straight, central cut along cutting line 180, from above the nose to the back of the head, is desired, the cutting device 50 is moved in this direction using the handle 80. During cutting of an arced material, the lower guide surface 160 at the vertex remains tangent with an upper skull surface of the adjacent skull 214 adjacent to the vertex of the cutting edge 100. Additional forked cuts may be made in the skull 214 at lines 200, 210. The cutting device 50 may be moved along cutting line 190 if a circular cut through the skull 214 around the periphery of the brain 216 is desired for accessing the brain 216 for experimentation.

It should be noted, the cutting device 50 (and the cutting devices described below) or one or more elements of the cutting devices may be used in an automated process for cutting biological tissue at a controlled depth. For example, in the exemplary application of at least partially removing a skull from a juvenile animal, the method may be automated by providing the head of the juvenile animal in a fixed position; providing an automatic cutting device including a body, a blade 90 with a cutting edge 100 coupled to body, and a guide surface 160 opposite of and fixed relative to the cutting edge 100; automatically inserting the cutting edge 100 of the cutting device through the skull of the top of the fixed head of the juvenile animal without penetrating the underlying brain; automatically moving the cutting device along the skull so that the cutting edge 100 cuts the skull without penetrating the underlying brain and the guide surface maintains a tangent orientation with respect to the adjacent skull adjacent to the vertex of the cutting edge 100; and automatically at least partially separating the skull from the underlying brain to access the brain of the juvenile animal.

With reference to FIGS. 3-5 and 7-14, and initially FIG. 3, a number of additional embodiments of a cutting device for cutting biological tissue or other material at a controlled depth to prevent damage to underlying tissue or object will be described. Similar elements in the Figures will be shown with like reference numbers, but with a different suffix (e.g., a, b, c, etc.). In FIG. 3, an alternative embodiment of a cutting device 220 is shown. The cutting device 220 is similar to the cutting device 50 illustrated in FIGS. 1, 2, and 6, except the cutting device 220 includes a blade 90a instead of cutting member 60. Similar to the blade 90 of FIGS. 1 and 2, the blade 90a includes an upper cutting edge 100a, a bottom dull edge 110a, and a sharp, pointed distal tip 120a. The cutting edge 100a and guide surface 160a intersect at a vertex 170a to form a cutting angle. Similar to the cutting device 50, the rear portion 130a of the guide member 70a preferably includes a handle with a holding portion for providing a line of force tangent to the skull 214 at the point of the leading edge of the cut and controlling movement of the cutting device 50. The blade 90a may be permanently fixed relative to the guide member 70a, or the blade 90a may be movable/adjustable relative to the guide member 70a to adjust the depth of the cutting edge 100a, the cutting angle of the cutting edge 100a, and/or the position of the cutting edge 100a. The blade 90a may be integral with the guide member 70a or may be a separate element from the guide member 70a. The cutting device 220 may be one of a plurality of different cutting devices 220, each designed for a different cutting application. The different cutting devices 220 may have different blade constructions (e.g., one or more of different cutting edge depths, different cutting angles, and/or different cutting edge positions). The cutting device 220 may be sterilizable/autoclavable (e.g., made of metal such as stainless steel, titanium) or may be disposable (e.g., plastic guide member 70a with metal blade 90a). The cutting device 220 is used in a similar manner to cutting device 50 described above.

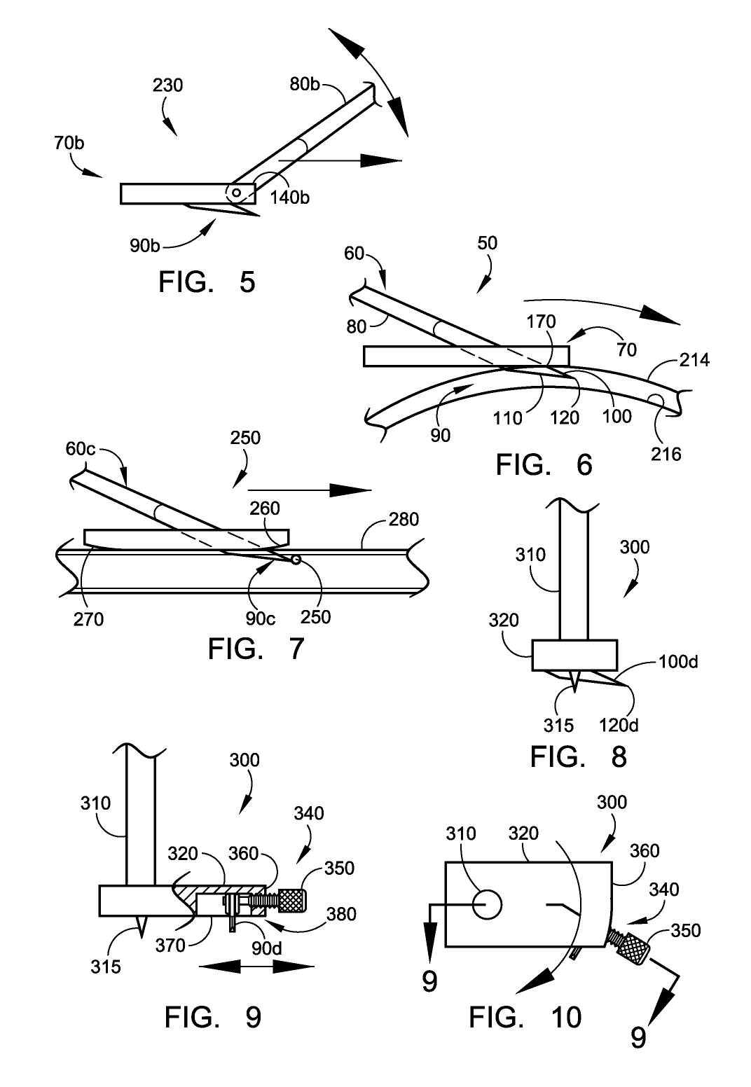

With reference to FIG. 4, another embodiment of a cutting device 230 will be described. The cutting device 230 is similar to the cutting device 220 described with respect to FIG. 3, except the cutting device includes a handle 80b angled forwardly, above the front portion 140b of the guide member 70b, towards the direction of travel of the cutting device 230. The handle 80b may be a separate member attached to the guide member 70b or may be integrated with the guide member 70b. Alternatively, the handle 80b may be part of the front portion 140b of the guide member. In FIG. 4, the handle 80b is fixed relative to the guide member 70b; however, with reference to FIG. 5, in an alternative embodiment of the cutting device 230, the handle 80b may be pivotally connected to the guide member 70b for pivotal movement of the handle 80b during use. The handle 80b forms a holding portion that may be gripped by a user to control movement of the cutting device 230. It should be noted, the cutting device 230 shown in FIG. 5 may include angular stops restricting the amount of angular pivoting movement allowed. The cutting device 230 in FIG. 5 may also including a locking mechanism for locking the handle 80b in fixed angular position relative to the guide member 70b prior to cutting. Providing a handle 80b oriented towards the direction of travel of the cutting device 230 allows a user to cut through biological tissue with a pulling action instead of the general pushing action used in controlling the cutting devices 50, 220 described above with respect to FIGS. 1-3. This causes the point of tangency to occur at the vertex, which is optimal, for a range of instrument orientations. With the pivot/pull design of cutting device 230 (FIG. 5), a user only needs to maintain an approximate orientation because the guide member 70b/blade 90b will automatically adjust such that the point of tangency is at the vertex. The pull design of cutting devices 230 (FIGS. 4, 5), especially, the pivot/pull design of cutting device 230 (FIG. 5) increases the speed of the cutting procedure and makes the cutting procedure less stressful mentally/physically to the user compared to the cutting devices 50, 220 described above.

With reference to FIG. 7, another embodiment of a cutting device 240 is shown. The cutting device 240 is generally similar to the cutting device 50 described above with respect to FIGS. 1 and 2, except the sharp, pointed tip 120c of the blade 90c is blunt or covered with a blunt tip cover 250 to prevent the possibility of the tip 120c accidentally penetrating biological tissue not intended to be cut. The blunt tip or blunt tip cover 250 may be a separate element from the blade 90c (e.g. a ball of epoxy, a soldered ball) or may be integrally formed with the rest of the blade 90c. Additionally, opposite rear end 260 and front end 270 of the lower guide surface 160c of the guide member 70c are angled upwardly, away from a biological tissue 280 to be cut, to prevent these ends 260, 270 from catching on the biological tissue 280 to be cut. In an alternative embodiment, only a lead end (e.g., front end 260) may be angled upwardly, away from a biological tissue 280. In use, an incision is made through the biological tissue 280 with a separate instrument and the blade 90c is inserted through the incision and advanced as shown in FIG. 7, cutting through the biological tissue 280. During cutting, the blunt tip cover 250 prevents the sharp tip 120c from cutting biological tissue not intended to be cut and the upwardly angled ends 260, 270 prevent the lower guide surface 160c from catching on the biological tissue 280. The cutting device 240 is particularly well-suited for cutting tendon sheaths, blood vessels, and other lumen-forming body structures.

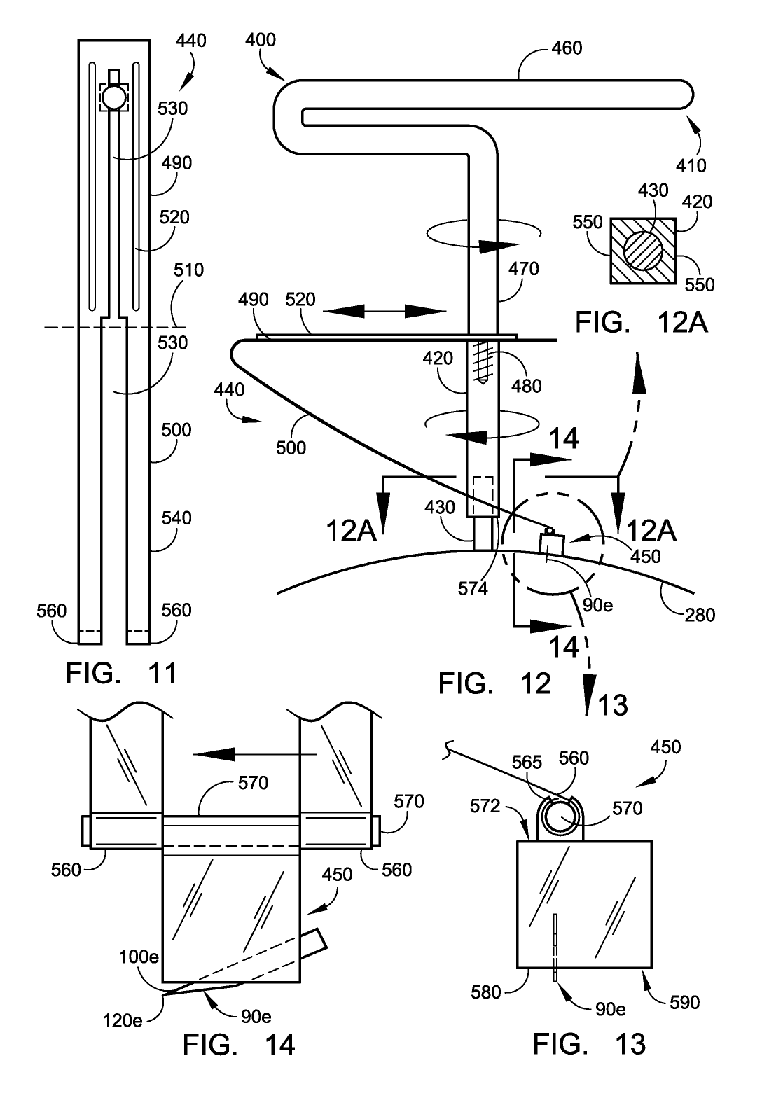

With reference to FIGS. 8-10, a further embodiment of a cutting device 300 will be described. The cutting device 300 is a rotatable cutting device 300 for making circular cut-outs in a material, preferably a flat material (e.g., biological tissue or other material). The cutting device 300 includes a shaft 310, a tip 315, a guide member in the form of a cutting block 320 extending from a lower portion of the shaft, and a blade 90d extending downwardly from the cutting block 320. Although not shown, an upper part of the shaft 310 may include a holding portion such as, but not limited to, that shown and described below in FIG. 12. The cutting device 300 may include a blade radial distance adjustment mechanism 340. In the embodiment shown, the blade radial distance adjustment mechanism 340 includes a radius control screw 350 threadably engaged in an end 360 of the cutting block 320. An end of the radius control screw 350 is coupled with the blade 90d so that clockwise and counterclockwise movement of the radius control screw 350 moves the blade 90d radially in or out for controlling the radius of the circular cut-out. The tip 315 may be pointed as shown in FIGS. 8 and 9, may be a two-part swivel where an upper part rotates with rotation of the shaft 310 and a lower part does not move relative to the material it engages, may be externally threaded (e.g., a screw), and/or may be fixed to the material to be cut with an adhesive (e.g., cyanoacrylate).

In use, the tip 315 of the cutting device 300 is placed on the material to be cut, at the center of the desired circular cut-out. The sharp, pointed distal tip 120d is inserted through the material to be cut. Upon insertion of the blade 90d and during use of the cutting device 300, a lower guide surface 370 of the cutting block 320 prevents the distal tip 120d from extending too deep below the material being cut, preventing damage to an underlying object. After inserting the distal tip 120d and cutting edge 100d through the material to be cut, the shaft 310 is rotated in the direction of the arrow shown in FIG. 10. During rotational cutting, the lower guide surface 370 remains tangent with an upper surface of the adjacent material being cut adjacent to the vertex of the cutting edge 100d. An outer portion 380 of the guide surface 370, which extends radially beyond the blade 90d and circular cut, slides along the material surface radially beyond the circular cut, which may help prevent the cutting device 300 from pushing into and damaging an underlying object below the circular cut-out during rotational cutting.

With reference to FIGS. 11-14, an alternative embodiment of a rotational cutting device 400 is shown. The rotational cutting device 400 includes a handle member 410, a shaft 420, a pivot pin 430, a spring mechanism 440, a guide member in the form of a cutting block 450, and a cutting blade 90e. The handle member 410 includes an upper grip portion 460 and a lower shaft portion 470. The handle member 410 forms a holding portion that may be gripped by a user to control rotational movement of the cutting device 400. An externally threaded member 480 extends downwardly from the lower shaft portion 470 and threadably engages an internally threaded portion of the shaft 420 for connecting the handle member 410 to the shaft 420. In the embodiment shown in FIG. 12, the shaft 420 has a square cross-section; however, in an alternative embodiment, the shaft 420 may have a rectangular cross-section. The pivot pin 430 is slidably disposed with a bottom of the shaft 420. A bottom of the pivot pin 430 may be affixed to the biological tissue 280 with an adhesive (e.g., dental cement, cyanoacrylate). The handle member 410 and the shaft 420 rotate with each other and rotate relative to the pivot pin 430, which is preferably affixed to the biological tissue 280. In alternative embodiments, the pivot pin 430 may be a different type of tip such as, but not limited to, a screw tip and a needle tip.

The spring mechanism 440 may be made of a metal such as, but not limited to, stainless steel and includes a flat, laterally extending upper portion 490 and an angularly extending lower portion 500 separated at bend line 510, where the spring mechanism 440 is bent. The upper portion 490 includes stiffening ribs 520 to impart stiffness in the upper portion 490 and an elongated recess 530 that slidingly receives the externally threaded member 480 for adjusting the radial distance of the blade 90e. The lower portion 500 of the spring mechanism 440 includes parallel arms 540 that straddle opposite faces 550 of the shaft 420 to provide the necessary torque for rotating the blade 90e to create a circular cut-out in the biological tissue. The arms 540 terminate in respective collars 560.

The collars 560 and a receptacle 565 on the cutting block 450 receive a connection pin 570 for connecting the cutting block 450 to the spring mechanism 440. The connection pin 570 snaps into the receptacle 565 and is fixed to the arms 540 of the spring mechanism 440 by being gripped by the collars 560. The fit between the connection pin 570 and the receptacle 565 is loose enough to allow the cutting block 450 to rotate about the connection pin 570. The connection pin 570 stabilizes the arms 540, preventing the arms 540 from twisting/springing out of shape while the user applies torque to advance the cut. The connection pin 570 also allows the cutting block 450 to rotate about the center of the pivot pin 430, thereby allowing the cutting block 450 to remain tangent to the skull at the vertex of the cutting angle. The receptacle 565 may be integral with the cutting block 450 (e.g., cast in plastic as one piece) or the receptacle 565 may be of a completely different design (e.g., as a spring leaf that keeps the connection pin 570 pressed in place). Alternatively, the connection pin 570 may be embedded permanently or non-permanently into the cutting block 450. In the event the connection pin 570 is embedded permanently or non-permanently into the cutting block 450, the collars 560 snap onto the connection pin 570, and the arms 540 are stabilized by some other cross member.

Adjacent the connection pin 570, the cutting block 450 includes a shelf 572 that may mate with a lower end 574 of the shaft 420 when the cutting block 450 is moved radially inward against the pivot pin 430. The blade 90e is oriented generally below the connection between the cutting block 450 and the spring mechanism 440, and preferably below and slightly to the left of this connection. A lower guide surface 580 of the cutting block 450 includes an outer portion 590, which extends radially beyond the blade 90d and circular cut.

In use, an adhesive is added to the bottom of the pivot pin 430, the pivot pin 430 is adhered to the biological tissue 280, at the center of the desired circular cut-out, and the shaft 420 is inserted over the pivot pin 430. The sharp, pointed distal tip 120e of the blade 90e is inserted through the biological tissue 280. Upon insertion of the blade 90e and during use of the cutting device 400, the lower guide surface 580 of the cutting block 450 prevents the distal tip 120e from extending too deep below the biological tissue being cut, to prevent damage to underlying tissue. After inserting the distal tip 120e and cutting edge 100e through the biological tissue, the handle member 410 is rotated. The spring mechanism 440 translates the rotational movement of the handle member 410 to rotational movement of the cutting blade 90. Rotational movement of the handle member 410 twists and torques spring mechanism 440, causing the cutting blade 90e to rotate and cut a circular cut-out in the biological tissue surface 280. During rotational cutting, the lower guide surface 580 remains tangent with an upper surface of the adjacent biological tissue 280 adjacent to the vertex of the cutting edge 100e. The spring mechanism 440 allows the cutting block 450 to rise and fall with variations in skull height encountered as the circular cut progresses, allows the user to be less precise in holding the shaft 420 perpendicular at the pivot pin 430 to the biological tissue surface 280, and urges the cutting block 450 and the cutting blade 90e downward to facilitate the rotational cutting. The outer portion 590 of the guide surface 580, which extends radially beyond the blade 90e and circular cut, slides along the more stable biological tissue 280 radially beyond the circular cut and helps prevent the cutting device 400 from pushing into underlying tissue and causing damage to underlying tissue below the circular cut-out during rotational cutting. The radial distance of the blade 90e may be adjusted by slightly unscrewing the handle member 410 from the shaft 420, and then moving the threaded member 480 within the elongated recess 530 until the blade 90e is at the desired radial distance. In this manner, different radii circular cut-outs may be made in the biological tissue 280.

The embodiments of the cutting device shown and described herein are advantageous in that they allow a user to cut biological tissue or other materials at a controlled depth with damaging underlying tissue or objects. In biological tissue cutting applications, this not only makes more efficient use of biological specimens, but decreases the amount of time it takes to perform biological cutting operations and improves the repeatability and reliability of cutting operations.

It will be readily apparent to those skilled in the art that still further changes and modifications in the actual concepts described herein can readily be made without departing from the spirit and scope of the invention as defined by the following claims.

What is claimed is:

1. A cutting device for cutting through biological tissue at a controlled depth without damaging underlying tissue, comprising:
a holding member to be held by a user for operating the cutting device;
a cutting blade coupled to the holding member, the cutting blade including a cutting edge for cutting through the biological tissue;
a guide surface opposite of and fixed relative to the cutting edge to maintain a controlled cutting depth without damaging underlying tissue.

2. The cutting device of claim 1, wherein the cutting edge and the guide surface together form a cutting angle with a vertex, the cutting angle sized so that the guide surface remains tangent with the biological tissue adjacent to the vertex during cutting.

3. The cutting device of claim 2, wherein the cutting angle is less than 90 degrees.

4. The cutting device of claim 1, wherein the cutting blade includes a distal tip, and the distal tip is separated from the guide surface a distance where the distal tip does not contact the underlying tissue.

5. The cutting device of claim 4, wherein the distal tip points in the direction of the holding member, and the cutting device is pulled using the holding member.

6. The cutting device of claim 4, wherein the distal tip points away from the direction of the holding member, and the cutting device is pushed using the holding member.

7. The cutting device of claim 1, further including a guide member having the guide surface, and the holding member is pivotally coupled to the guide member.

8. The cutting device of claim 1, further including a guide member having the guide surface and the holding member.

9. The cutting device of claim 1, wherein the cutting device includes a pivot point, the cutting blade is disposed a radial distance from the pivot point, and the holding member is rotatable for rotating the cutting blade to create a circular cut-out in the biological tissue.

10. The cutting device of claim 9, wherein the pivot point is a swivel with a lower portion that is fixed to the biological tissue during rotation of the holding member.

11. The cutting device of claim 9, wherein the pivot point is a pin that rotates relative to the biological tissue during rotation of the holding member.

12. The cutting device of claim 9, wherein the pivot point is a pin that is fixed relative to the biological tissue during rotation of the holding member, and the holding member rotates relative to the pin.

13. The cutting device of claim 9, wherein the pivot point is a screw.

14. The cutting device of claim 9, wherein the radial distance between the cutting edge and the pivot point is adjustable for providing different radius circular cut-outs in the biological tissue.

15. The cutting device of claim 1, wherein the cutting blade is replaceable with same or a different cutting blade.

16. The cutting device of claim 1, further including a guide member having the guide surface, and the guide member is replaceable with the same or different guide members.

17. The cutting device of claim 1, wherein the cutting blade is adjustable for different biological tissue cutting applications.

18. The cutting device of claim 1, wherein the cutting device is one of a plurality of cutting devices of a kit, the plurality of cutting devices include at least one of different cutting edges, different cutting edge angles, different cutting edge depths, and different holding member configurations.

19. A method of use of the cutting device of claim 1, comprising using the cutting device to cut through biological tissue at a controlled depth without damaging underlying tissue.

20. The method of claim 19, wherein the biological tissue is a soft skull of a juvenile animal, and the method includes cutting through the soft skull of the juvenile animal to access a brain of the juvenile animal without damaging the underlying brain.

21. A method of cutting through biological tissue at a controlled depth without damaging underlying tissue, comprising:
providing a cutting device including a holding member to be held by a user, a cutting blade coupled to the holding member, the cutting blade including a cutting edge, cutting angle, and vertex, and a guide surface opposite of and fixed relative to the cutting edge;
inserting the cutting edge of the cutting device through the biological tissue;
moving the cutting device so that the cutting edge of the blade cuts the biological tissue without damaging underlying tissue and the guide surface remains tangent with the biological tissue adjacent to the vertex.

22. A cutting device, comprising:
a holding member to be held by a user for operating the cutting device;
a cutting blade coupled to the holding member, the cutting blade including a cutting edge;
a guide surface opposite of and fixed relative to the cutting edge to maintain a controlled cutting depth.

23. A method of cutting through an object at a controlled depth, comprising:
providing a cutting device including a holding member to be held by a user, a cutting blade coupled to the holding member, the cutting blade including a cutting edge, cutting angle, and vertex, and a guide surface opposite of and fixed relative to the cutting edge;
at least one of inserting the cutting edge of the cutting device through the object or positioning the cutting edge of the cutting device adjacent an edge of the object;
moving the cutting device so that the cutting edge of the blade cuts the object and the guide surface remains tangent with the object adjacent to the vertex.
